(19) United States
(12) Patent Application Publication
(10) Pub. No.: US 2007/0203534 A1
(43) Pub. Date: Aug. 30, 2007

(54) Stimulating galvanic or slow AC current for therapeutic physiological effects

(76) Inventor: Robert Tapper, Los Angeles, CA (US)

Correspondence Address: FULWIDER PATTON LLP, HOWARD HUGHES CENTER, 6060 CENTER DRIVE, TENTH FLOOR, LOS ANGELES, CA, 90045, US

(21) Appl. No.: 11/651,931

(22) Filed: Jan. 10, 2007

Related U.S. Application Data
(60) Provisional application No. 60/772,762, filed on Feb. 13, 2006.

(51) Int. Cl. A61N 1/32; A61N 1/00

(52) U.S. Cl. 607/50; 607/76

(57) ABSTRACT

Basically, the present invention is directed to a new and improved system for the therapeutic use of currents which includes conducting direct electrical current through the skin of a body being treated, and periodically reversing the electrical current and conducting the current through the skin in the opposite direction, to effectively deliver very low frequency AC current, substantially in the critical range of approximately 0.0027 Hz to 20 Hz. It has been discovered that, within this substantially critical frequency window between approximately six minutes per full cycle and approximately ten cycles per second, a dramatic cancellation of skin damaging ions takes place. At frequencies higher than approximately 20 Hz, the effect is to diminish its DC-like blood stimulation. At frequencies lower than approximately 0.0027 Hz, the risk of skin injury increases substantially. It is well known that the positive electrode unfortunately produces skin damaging hydrochloric acid. Likewise, the negative electrode unfortunately also produces skin damaging sodium hydroxide. However, within the aforementioned frequency range of the present invention, either polarity stimulates blood circulation, but also cancels the undesired skin damaging ions with the reverse portion of the electrical cycle. The reason for neutralization of the harsh injury producing chemicals, i.e., hydrochloric acid and sodium hydroxide, is that both of these chemicals require a finite period of time on the skin to cause damage. Hence, these damaging chemicals are made to cancel each other before damage takes place, by critical frequency selection, in accordance with the invention, of the AC driving signal. Therefore, optimization of a long sought electrotherapeutic device with reduced side effects has been achieved. Another use of the safe AC currents cited above and/or a DC signal with charged membranes preventing injury is its application to wound healing. The conductive electrodes for these devices may take either of two forms, i.e., one may be non-metallic carbon-filled silicone or, preferably of powdered carbon particles. A second form may be a metallic electrode preferably of aluminum, copper, zinc and/or magnesium as examples of metallic electrodes but not necessarily limited to these metals. These metals are preferably in powdered form and contained within a porous membrane with a small opening to attach a conductive lead to a battery source. Still other applications of the innovative use in electrotherapy of charged membranes and/or powdered metal electrodes is its use for drug delivery and diagnostic purposes. For instance, a membrane enclosed stainless steel powdered negative electrode may be used in the pickup probe for glucose detection. Charged membranes would surround the probe as an intervenor between skin and the electrode.

### CROSS-REFERENCES TO RELATED APPLICATIONS

This application is claiming the benefit of Provisional Application No. 60/772,762 filed Feb. 13, 2006. The following documents are incorporated herein by reference: United States Publication No. US 2005/0192528 A1, Publication Date Sep. 1, 2005 entitled METHODS, APPARATUS AND CHARGED CHEMICALS FOR CONTROL OF IONS, MOLECULES OR ELECTRONS; United States Patent Publication No. US 2006/0025714 A1, Publication Date Feb. 2, 2006 entitled METHODS, APPARATUS AND CHARGED CHEMICALS FOR CONTROL OF IONS, MOLECULES OR ELECTRONS; U.S. Pat. No. 5,224,927 issued Jul. 6, 1993 entitled IONTOPHORETIC TREATMENT SYSTEM and U.S. Pat. No. 4,340,047 issued Jul. 20, 1982 entitled IONTOPHORETIC TREATMENT APPARATUS.

This invention relates generally to improvements in electrotherapeutic systems and, more particularly, to stimulating galvanic or slow AC current for therapeutic physiological effects.

### BACKGROUND OF THE INVENTION

Medical galvanism or slow AC current has different application and effects compared to iontophoretic devices which are intended to infuse ionic medication or drugs and is wholly dependent on the polarity at that moment. The galvanic current causes vasomotor stimulation of the skin and increased local blood circulation and nutrition of the parts between the electrodes irrespective of polarity. The generally accepted explanation of the therapeutic effect is that the improvement of blood circulation speeds up resorption of inflammatory products, reflex stimulation or relief of pressure and relieves pain. In addition, local and general function may be appreciably stimulated. The galvanic current has proven clinically useful in a number of acute and chronic inflammatory conditions, such as: (1) selected cases of arthritis and rheumatic conditions, neuritis and neuralgia, mostly in the chronic stage; (2) selected cases of traumatism, contusions, sprains, myositis, both in the acute and in the chronic stage.

Accordingly, there has been a long existing need for improved electrotherapeutic stimulating systems.

### SUMMARY OF THE INVENTION

Basically, the present invention is directed to a new and improved system for the therapeutic use of currents which includes conducting direct electrical current through the skin of a body being treated, and periodically reversing the electrical current and conducting the current through the skin in the opposite direction, to effectively deliver very low frequency AC current, substantially in the critical range of approximately 0.0027 Hz to 20 Hz. It has been discovered that, within this substantially critical frequency window between approximately six minutes per full cycle and approximately ten cycles per second, a dramatic cancellation of skin damaging ions takes place. At frequencies higher than approximately 20 Hz, the effect is to diminish its DC-like blood stimulation. At frequencies lower than approximately 0.0027 Hz, the risk of skin injury increases substantially. It is well known that the positive electrode unfortunately produces skin damaging hydrochloric acid. Likewise, the negative electrode unfortunately also produces skin damaging sodium hydroxide. However, within the aforementioned frequency range of the present invention, either polarity stimulates blood circulation, but also cancels the undesired skin damaging ions with the reverse portion of the electrical cycle. The reason for neutralization of the harsh injury producing chemicals, i.e., hydrochloric acid and sodium hydroxide, is that both of these chemicals require a finite period of time on the skin to cause damage. Hence, these damaging chemicals are made to cancel each other before damage takes place, by critical frequency selection, in accordance with the invention, of the AC driving signal. Therefore, optimization of a long sought electrotherapeutic device with reduced side effects has been achieved. Another use of the safe AC currents cited above and/or a DC signal with charged membranes preventing injury is its application to wound healing. The conductive electrodes for these devices may take either of two forms, i.e., one may be non-metallic carbon-filled silicone or, preferably of powdered carbon particles. A second form may be a metallic electrode preferably of aluminum, copper, zinc and/or magnesium as examples of metallic electrodes but not necessarily limited to these metals. These metals are preferably in powdered form and contained within a porous membrane with a small opening to attach a conductive lead to a battery source. Still other applications of the innovative use in electrotherapy of charged membranes and/or powdered metal electrodes is its use for drug delivery and diagnostic purposes. For instance, a membrane enclosed stainless steel powdered negative electrode may be used in the pickup probe for glucose detection. Charged membranes would surround the probe as an intervenor between skin and the electrode.

These and other objects and advantages of the invention will become apparent from the following more detailed description, when taken in conjunction with the accompanying drawings of an illustrative embodiment.

### BRIEF DESCRIPTION OF THE DRAWINGS

FIG. 1 is an internal perspective view of an osteoarthritic knee with the system of the present invention installed for treatment;

FIG. 2 is an external perspective of a treatment pad/electrode held against the skin of the subject by a support sock;

FIG. 3 is a perspective view of the electronic components installed on the treatment subject; and

FIGS. 4-1 through 4-9 are an electrical schematic of circuitry suitable for practice of the invention.

### DETAILED DESCRIPTION OF THE PREFERRED EMBODIMENTS

Investigations have shown that a galvanic treatment of sufficient intensity exerts vasomotor stimulation lasting for several hours. Since the effect at each pole is about the same, no special attention has to be paid to the direction of current flow or polarity. This therefore, equates to a very slow AC signal which while behaving like DC, does not have its problems. The value of an AC signal over DC is apparent when one considers that a DC signal will develop skin injury producing chemicals. The positive pole sends hydrochloric acid to the skin causing irritation and the negative pole sends sodium hydroxide to the skin causing burns and possible permanent scarring. With the use of a slow AC signal at a frequency slow enough to mimic the DC therapeutic effect, the unwanted acid and alkaline chemicals are neutralized. Another innovation which makes a DC signal usable without the aforementioned skin injury problems is the use of charged membranes, such as those described in the aforedescribed applications, to prevent the hydrochloric acid ions and the sodium hydroxide ions from reaching the skin. The charged membranes are placed between the electrode and the skin and also act as a water reservoir for electrical conductivity between these two points. Another preventative of skin injury in a DC device would be to use aluminum powder on the positive electrode and stainless steel on the negative electrode. A typical use for the system using either a slow AC or DC signal would be for relief of pain of an osteoarthritic knee joint. Literature indicates a patient with this problem lacks a normal blood flow in this area leading to loss of cartilage with resulting pain. Application of this device with either current and with the aforementioned protective charged membranes will restore blood flow, reduce pain and not incur skin injury. Other applications would be treatments of carpal tunnel syndrome, wound healing, decubitus ulcers, tremors, diabetic feet and any other problems that would benefit from increased blood circulation. The conductive electrodes for these devices may take either of two forms, i.e., one may be non-metallic carbon-filled silicone or, preferably of powdered carbon particles. A second form may be a metallic electrode preferably of aluminum, copper, zinc and/or magnesium as examples of metallic electrodes but not necessarily limited to these metals. These metals are preferably in powdered form and in a mixture with a saline gel.

The present invention includes shock and pain prevention which may be further enhanced by slow ramp up and/or down of the applied electrical current (e.g., see U.S. Pat. No. 4,340,047), and/or a new and improved system for the therapeutic use of currents which includes conducting direct electrical current through the skin of a subject being treated, and periodically reversing the electrical current and conducting the current through the skin in the opposite direction, to effectively deliver very low frequency AC current, substantially in the critical range of approximately 0.0027 Hz to 20 Hz. It has been discovered that, within this substantially critical frequency window between approximately six minutes per full cycle and approximately ten cycles per second, a dramatic cancellation of skin damaging ions takes place. At frequencies higher than approximately 20 Hz, the effect is to diminish its DC-like blood stimulation. At frequencies lower than approximately 0.0027 Hz, the risk of skin injury increases substantially. It is well known that the positive electrode unfortunately produces skin damaging hydrochloric acid. Likewise, the negative electrode unfortunately also produces skin damaging sodium hydroxide. However, within the aforementioned frequency range of the present invention, either polarity stimulates blood circulation, but also cancels the undesired skin damaging ions with the reverse portion of the electrical cycle. The reason for neutralization of the harsh injury producing chemicals, i.e., hydrochloric acid and sodium hydroxide, is that both of these chemicals require a finite period of time on the skin to cause damage. Hence, these damaging chemicals are made to cancel each other before damage takes place, by critical frequency selection, in accordance with the invention, of the AC driving signal. Therefore, optimization of a long sought electrotherapeutic device with reduced side effects has been achieved. Another use of the safe AC currents cited above and/or a DC signal with charged membranes preventing injury is its application to wound healing. The conductive electrodes for these devices may take either of two forms, i.e., one may be non-metallic carbon-filled silicone or, preferably of powdered carbon particles. A second form may be a metallic electrode preferably of aluminum, copper, zinc and/or magnesium as examples of metallic electrodes but not necessarily limited to these metals. These metals are preferably in powdered form and contained within a porous membrane with a small opening to attach a conductive lead to a battery source. The powdered electrodes are desirably mixed with a saline gel. Still other applications of the innovative use in electrotherapy of charged membranes and/or powdered metal electrodes is its use for drug delivery and diagnostic purposes. For instance, a membrane enclosed stainless steel powdered negative electrode may be used in the pickup probe for glucose detection. Charged membranes would surround the probe as an intervenor between skin and the electrode.

The AC frequency may even be used for enhanced iontophoretic (drug delivery) purposes with the use of charged membranes or metal powder electrodes is included to prevent injury.

The DC unit of the present invention may also be used for drug delivery with novel protection against skin injury by the metal powders or charged membranes described above.

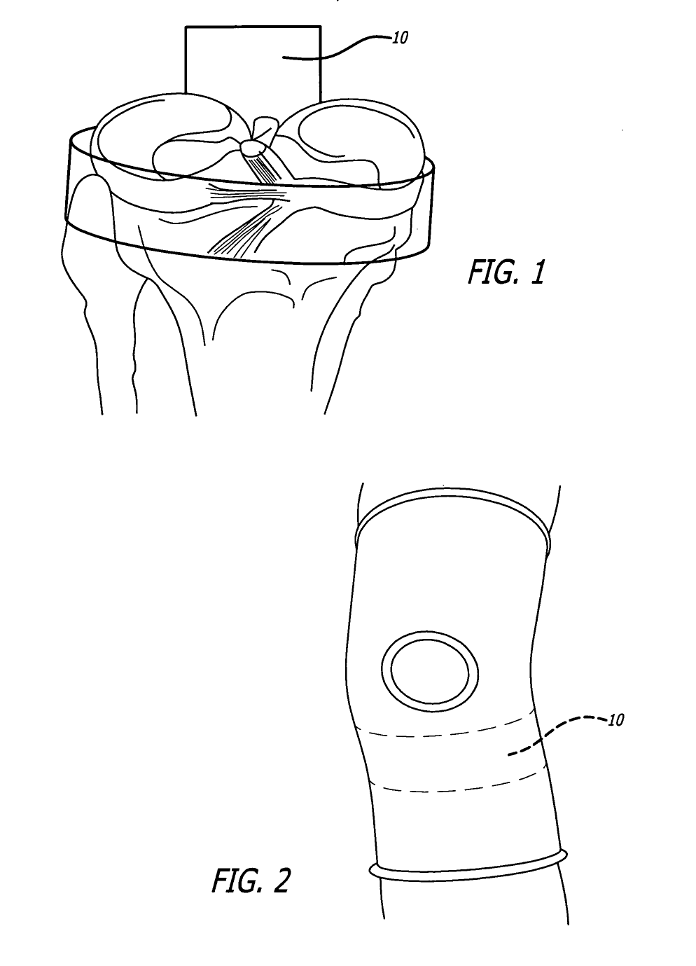

FIG. 1 shows wetted felt pads 10 between electrodes and skin to treat cartilage area.

FIG. 2 shows the treatment pad/electrode held against skin by a support sock (second pad/electrode on back of knee not shown).

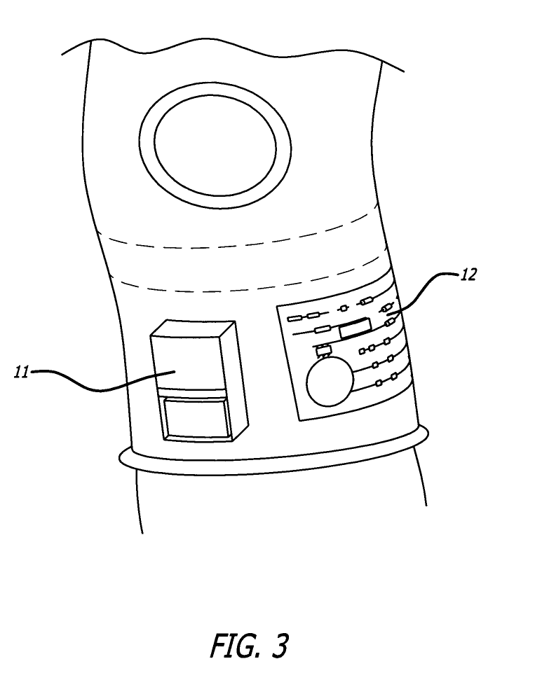

FIG. 3 shows electronic components 11 and 12 mounted outside the support sock but covered by pockets (not shown). Component 11 is a flat pack 9 volt battery or the like. Component 12 is a flexible circuit board for generating an AC signal.

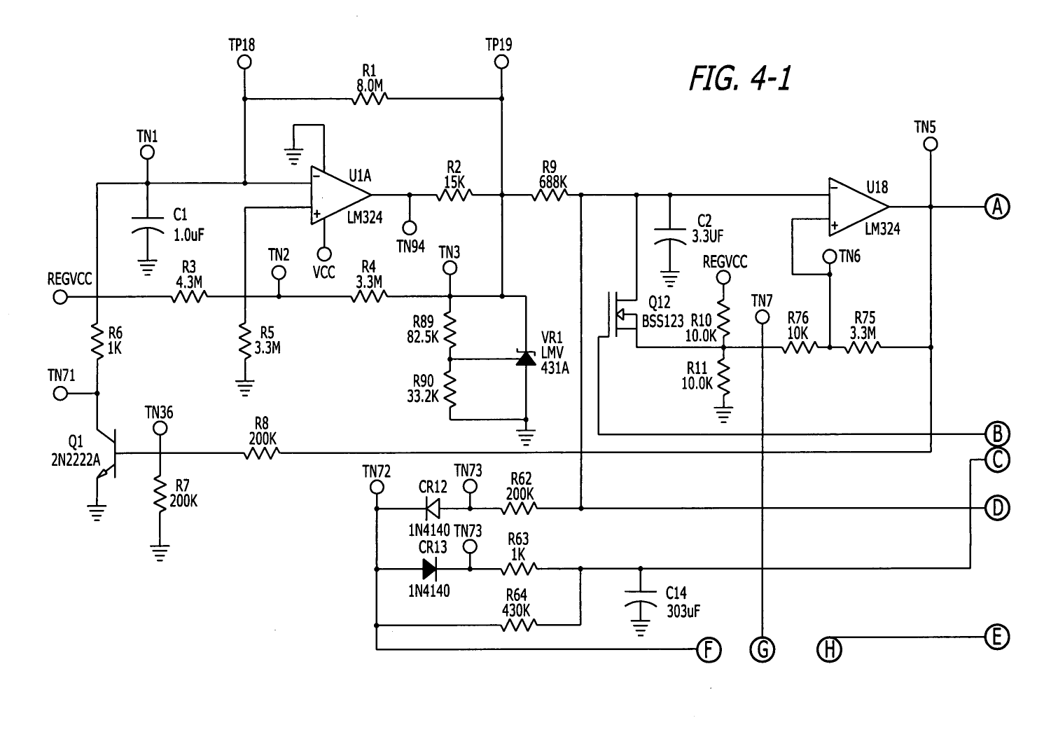

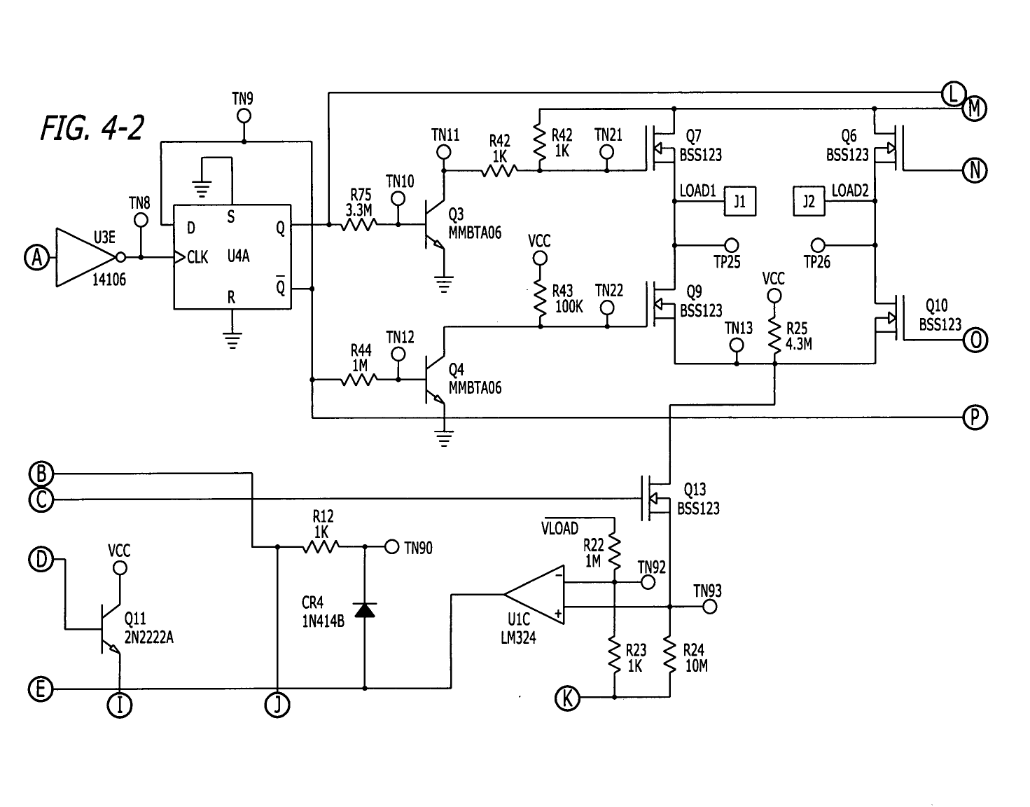

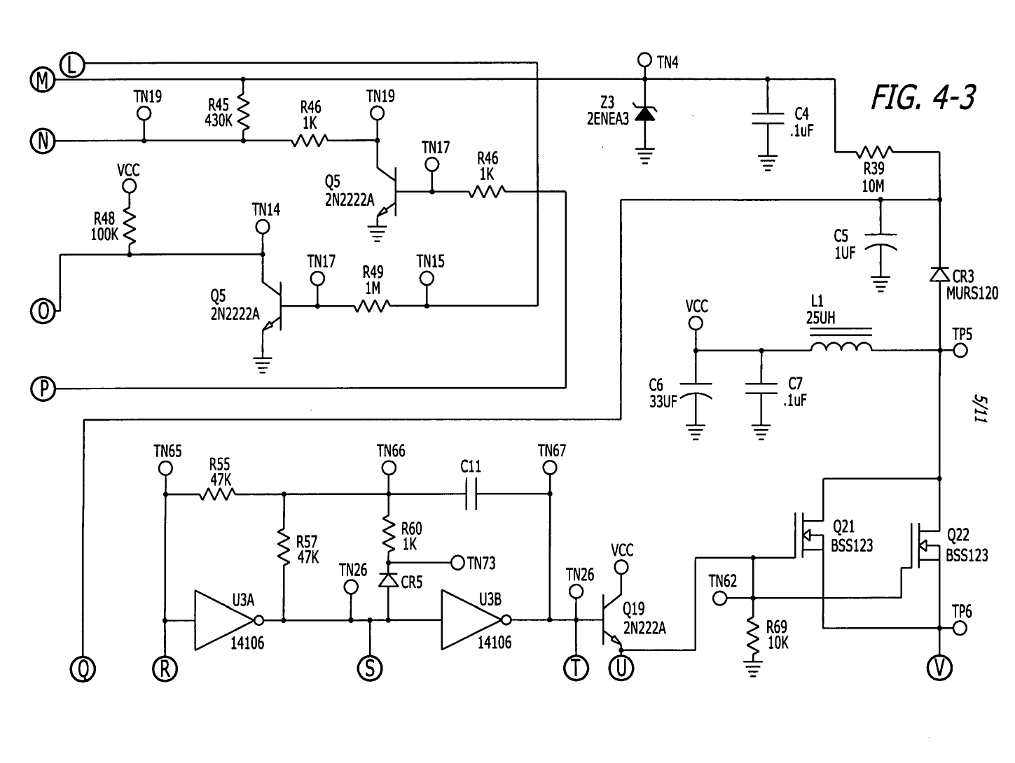

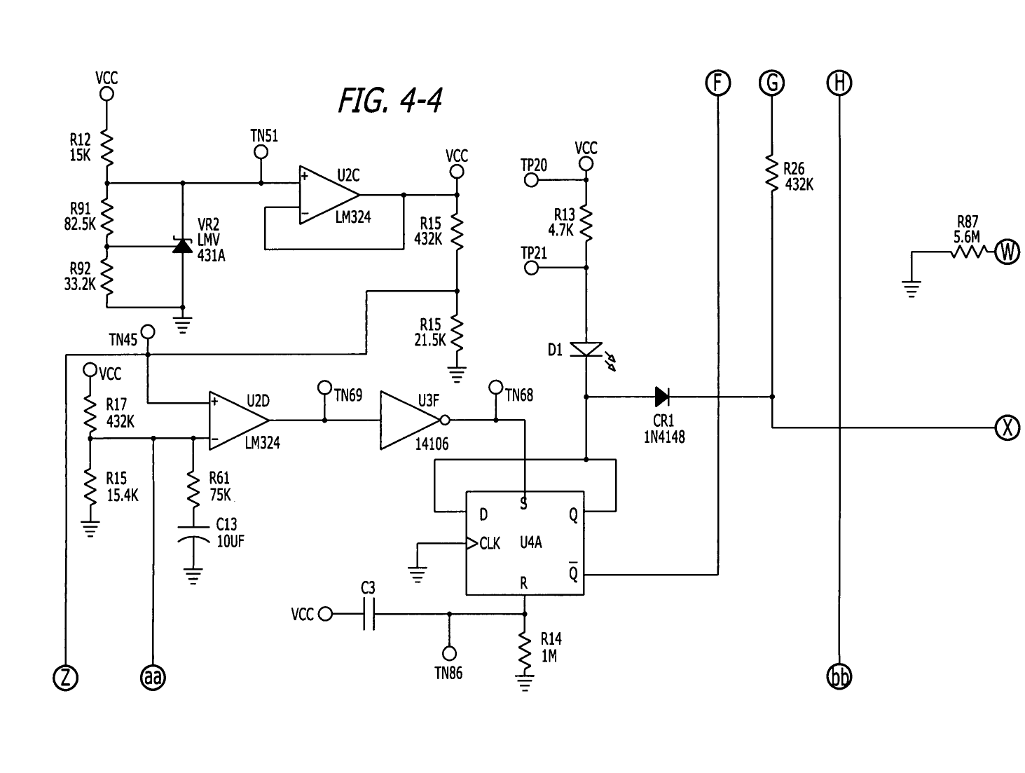

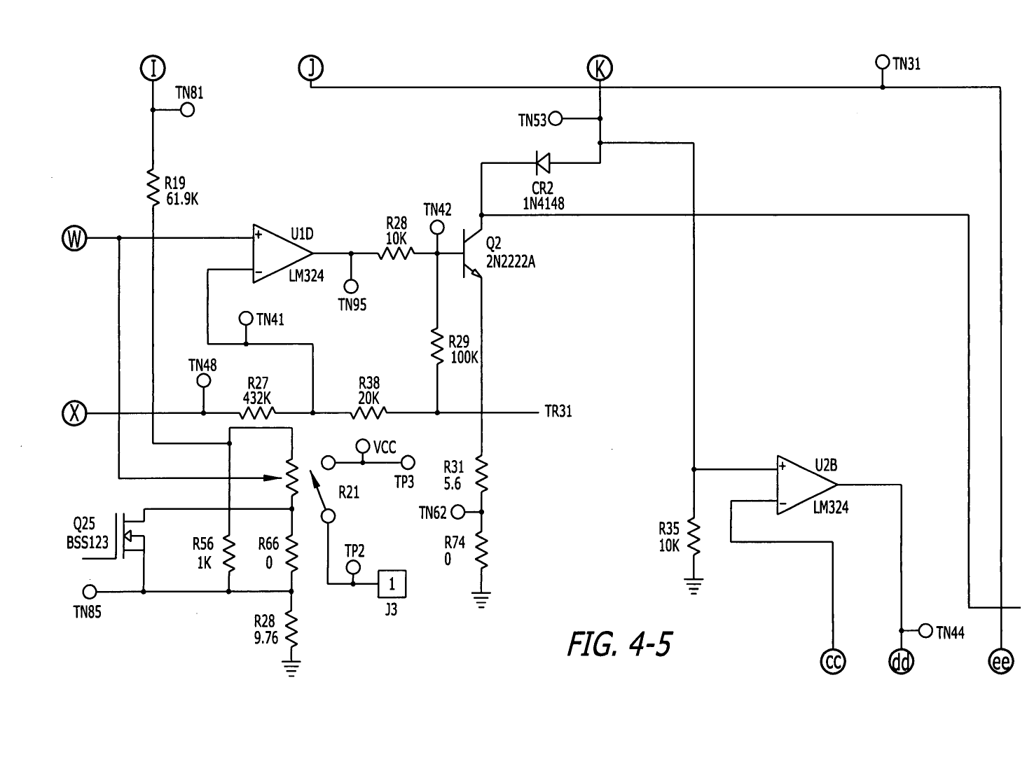

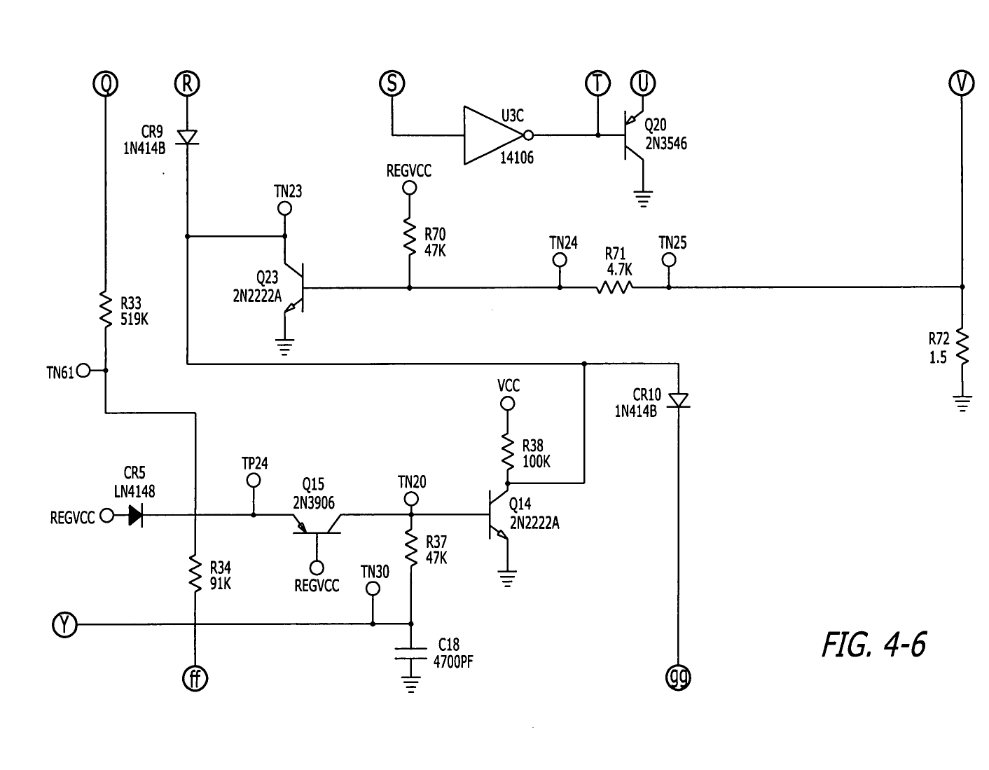

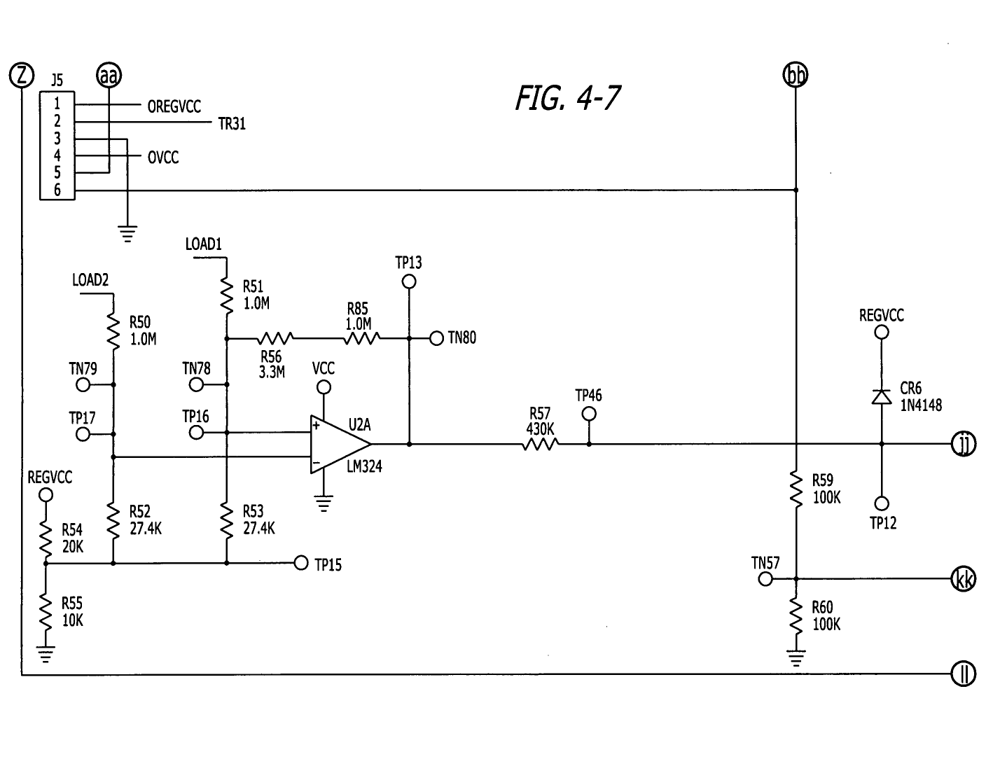

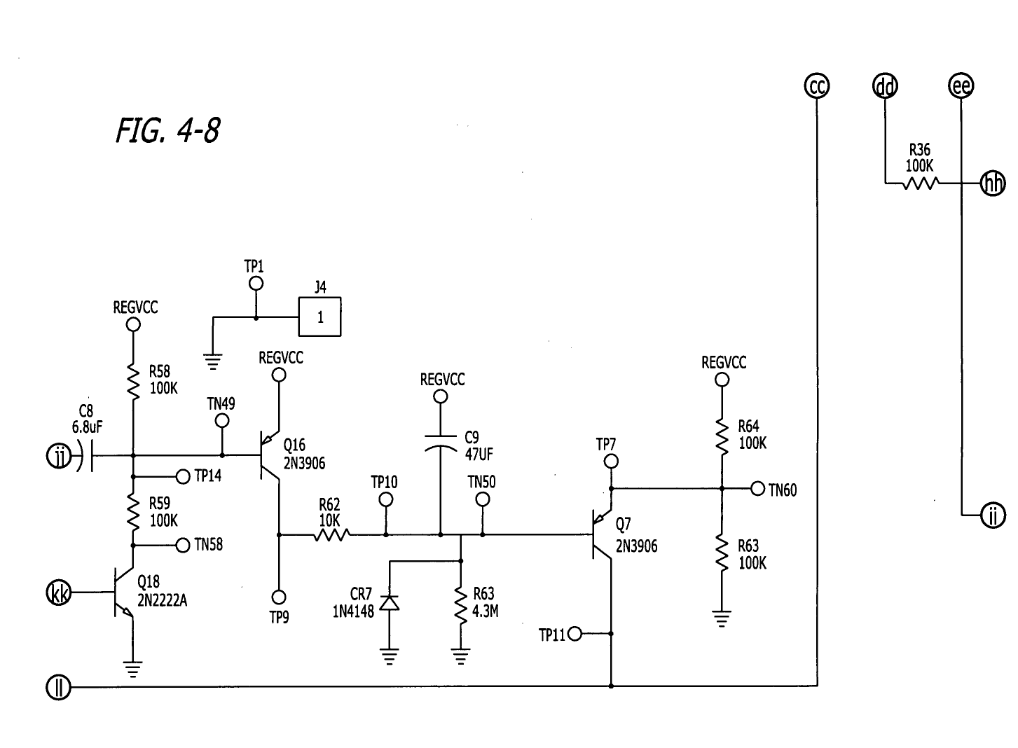

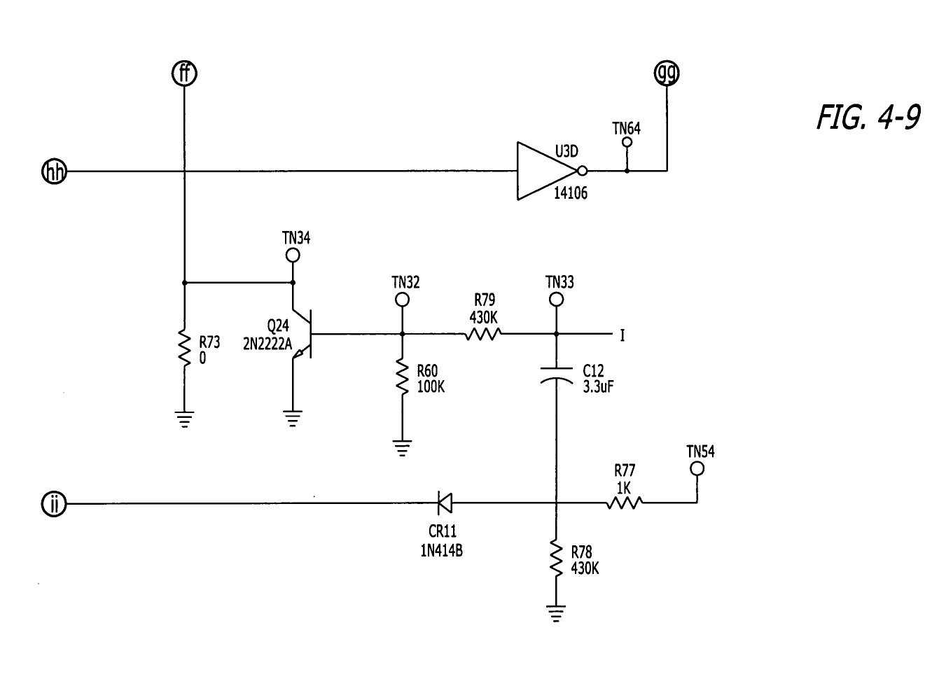

FIGS. 4-1 through 4-9 are an electric schematic for operating the system electronics.

Thus, improvements in therapeutic systems and, more particularly, to stimulating galvanic or slow AC current for therapeutic physiological effects is described in conjunction with one or more specific embodiments. It will be apparent from the foregoing that, while particular forms of the invention have been illustrated and described, various alternatives, modifications and variations can be made without departing from the spirit and scope of the invention. Accordingly, the invention is intended to embrace all such alternatives, modifications and variations and it is not intended that the invention be limited, except as by the appended claims.

I claim:

1. A method of applying electrical currents to a biological subject for electrotherapy, said method including the steps of:
conducting an electrical current through a surface of said subject in a first direction from a first electrode to a second electrode on said subject; and intermittently reversing, at a relatively low frequency which prevents skin damage, the polarity of said electrodes to cause said electrical current to flow in a second direction opposite to said first direction, whereby electrical currents may be continuous for extended periods of time and circulation is improved.

2. A method of applying electrical currents to a biological subject for electrotherapy, said method including the steps of:
conducting an electrical current through a surface of said subject in a first direction from a first electrode to a second electrode on said subject; and intermittently reversing, at a relatively low frequency which prevents skin damage, between approximately 20 times per second and approximately once every three minutes, the polarity of said electrodes to cause said electrical current to flow in a second direction opposite to said first direction, whereby electrical currents may be continuous for extended periods of time and circulation is improved.

3. A method as recited in either of claims 1 or 2 wherein a slow AC or DC signal supplies non-metallic conductive electrodes.

4. A method as recited in any of claims 1-3 wherein the electrodes are powdered carbon electrodes.

5. A method as recited in claim 4 wherein the carbon particles range in size from micron to nano.

6. A method as recited in any of claims 1-3 wherein the electrodes include copper particles.

7. A method as recited in any of claims 1-3 wherein the electrodes include a metal powder such as aluminum, copper, zinc, magnesium and/or stainless steel.

8. A method as recited in claim 7 wherein the metal powder is mixed with a saline gel.

9. In a method of applying electrical currents to a subject for electrotherapy, the steps of:
using charged membranes for solution storage and prevention of harmful chemicals emitted from electrodes from reaching the skin; and
a water solution between the electrode and skin to complete the circuit, whereby the device acts as stimulation to increase capillary blood flow in an osteoarthritic joint, typically the knees.

10. A method as recited in claim 9, wherein a DC electrical power source is connected to said electrodes.

11. A method as recited in any of claims 1-10, wherein the system is used for wound healing.

12. A method as recited in any of claims 1-10, wherein the system is used for restoring blood circulation.

13. A method as recited in any of claims 1-10, wherein the system is used for speeding tissue growth.

14. A method as recited in any of claims 1-13, wherein a slow AC or DC signal is also provided for bactericidal treatment.

15. A method for wound healing in a biological subject for electrotherapy, said method including the steps of:
conducting an electrical current through a surface of said subject in a first direction from a first electrode to a second electrode on said subject; and intermittently reversing, at a relatively low frequency which prevents skin damage, the polarity of said electrodes to cause said electrical current to flow in a second direction opposite to said first direction, whereby electrical currents may be continuous for extended periods of time and circulation is improved.

16. A method for restoring blood circulation to a biological subject for electrotherapy, said method including the steps of:
conducting an electrical current through a surface of said subject in a first direction from a first electrode to a second electrode on said subject; and intermittently reversing, at a relatively low frequency which prevents skin damage, the polarity of said electrodes to cause said electrical current to flow in a second direction opposite to said first direction, whereby electrical currents may be continuous for extended periods of time and circulation is improved.

17. A method for enhancing tissue growth in a biological subject for electrotherapy, said method including the steps of:
conducting an electrical current through a surface of said subject in a first direction from a first electrode to a second electrode on said subject; and intermittently reversing, at a relatively low frequency which prevents skin damage, the polarity of said electrodes to cause said electrical current to flow in a second direction opposite to said first direction, whereby electrical currents may be continuous for extended periods of time and circulation is improved.

18. A DC treatment device, comprising:
a powdered stainless steel negative electrode, said electrode being located within a porous membrane, whereby sodium hydroxide is prevented from reaching the skin of the subject being treated.

19. A DC treatment device, comprising:
a positive electrode utilizing powdered metals selected from aluminum, aluminum zirconium, copper, zinc, magnesium and the like, said electrode being located within a porous pouch to prevent hydrochloric acid from damaging the skin of the subject being treated.

20. In a method of applying electrical currents to a subject for electrotherapy, the step of:
using charged membranes for prevention of harmful chemicals emitted from electrodes from reaching the skin, whereby skin injury is mitigated.

21. A method as recited in claim 20, wherein the electrical current is AC utilized for drug delivery.

22. A method as recited in claim 20, wherein the electrical current is DC utilized for drug delivery.

23. In a method of applying electrical currents to a subject for electrotherapy, the step of:
using electrodes which include a metal powder such as aluminum, copper, zinc, magnesium and/or stainless steel, mixed with saline gel, for preventing skin injury.

24. A method as recited in claim 23, wherein the electrical current is AC utilized for drug delivery.

25. A method as recited in claim 23, wherein the electrical current is DC utilized for drug delivery.

26. A method as recited in any of the previous claims, wherein the stimulative electrical currents are applied therapeutically to benefit the problems of osteoarthritis,

27. A method as recited in any of the previous claims, wherein the stimulative electrical currents are applied therapeutically to benefit the problems of treating knee joints as well as other joints.

28. A method as recited in any of the previous claims, wherein the stimulative electrical currents are applied therapeutically to benefit the problems of carpel tunnel syndrome.

29. A method as recited in any of the previous claims, wherein the stimulative electrical currents are applied therapeutically to benefit the problems of decubitus ulcers.

30. A method as recited in any of the previous claims, wherein the stimulative electrical currents are applied therapeutically to benefit the problems of tremors.

31. A method as recited in any of the previous claims, wherein the stimulative electrical currents are applied therapeutically to benefit the problems of diabetic feet.

32. A method as recited in any of the previous claims, wherein the stimulative electrical currents are applied therapeutically to benefit the problems of aging skin.

33. A method as recited in any of the previous claims, wherein the stimulative electrical currents are applied therapeutically to benefit the problems of any other problems and/or disabilities that may benefit from increased blood circulation.

34. Each and every novel feature and/or novel combination of features herein disclosed.
